(19) United States
(12) Patent Application Publication
(10) Pub. No.: US 2017/0165093 A1
(43) Pub. Date: Jun. 15, 2017

(54) Leg Brace

(71) Applicants: Marc Lee Huttenlocker, Rocky Mount, MO (US); Martin Lee Huttenlocker, Western Springs, IL (US); Maurice Lee Huttenlocker, Columbia, SC (US); Matthew Lee Huttenlocker, SR., Troy, MO (US); Michael Lee Huttenlocker, JR., Troy, MO (US); John Robert Alexander, Troy, MO (US)

(72) Inventors: Marc Lee Huttenlocker, Rocky Mount, MO (US); Martin Lee Huttenlocker, Western Springs, IL (US); Maurice Lee Huttenlocker, Columbia, SC (US); Matthew Lee Huttenlocker, SR., Troy, MO (US); Michael Lee Huttenlocker, JR., Troy, MO (US); John Robert Alexander, Troy, MO (US)

(73) Assignee: H & A Foot Brace, LLC, Western Springs, IL (US)

(21) Appl. No.: 14/965,847

(22) Filed: Dec. 10, 2015

(51) Int. Cl. A61F 5/01

(52) U.S. Cl. CPC A61F 5/012; A61F 5/0111

(57) ABSTRACT

The current invention is a leg brace with two adjustable lateral supports connected to the footpad with a plurality of support straps that are connected between the lateral supports 10 and the lateral supports. The support straps can be attached around the leg that needs to be support with an attachment means such as a buckle mechanism or a hook and fastening system. The support straps would have built in one or more air bladders which would inflate with air using a built in air pump allowing the proper support and comfort for the user.

### CROSS-REFERENCES TO RELATED APPLICATIONS

None.

### STATEMENT AS TO RIGHTS TO INVENTIONS MADE UNDER FEDERALLY SPONSORED RESEARCH AND DEVELOPMENT

None

### FIELD OF THE INVENTION

This device refers to the field of medical support devices in more particularity Leg Braces that support an injured leg.

### BACKGROUND OF THE INVENTION

Leg injuries is one of the more common injuries that people can have. Especially lower leg injuries involving the foot and ankle.

Leg injuries occur millions of times every year. They are most common amongst athletes. The most concerning issue for most of these people is their inability to conduct their daily activities. Without the ability to walk and perform their daily tasks people become frustrated. There is an increased need for a product that can help those with leg injuries get back into the groove quicker. The medical market is only continuing to grow and thus the need for leg support will never become obsolete.

Currently crutches and the Air Cam Walker Fracture Boot are the two dominant products used medical supplies for leg injuries. The issue with crutches is that walking any distance begins to weaken the other leg since there is an imbalance. Then there is the fact that both hands are tied up using the crutches making it impossible to carry anything. Finally there is the issue of stairs; not only is it time consuming but also difficult to learn how to tackle the task.

With the Air Cam Walker, Some patients using the device have complained of having some redness on the ankle on the initial stages of using the device. In addition, there is not much padding where the heel strikes causing the heel to get sore if a patient tends to walk a lot.

There remains room for improvement in the art.

### SUMMARY OF THE INVENTION

This proposed device is a leg brace that can be used with either leg when that leg is injured.

The current invention is a leg brace with two adjustable lateral supports connected to a footpad with a plurality of support straps that are connected between the lateral supports.

These support straps can be attached around the leg that needs to be support with an attachment means such as a buckle mechanism or a hook and fastening system.

The footpad would have a rocker bottom to support a person's natural gait.

The support straps would have built in one or more air bladders which would inflate with air using a built in air pump allowing the proper support and comfort for the user.

### BRIEF DESCRIPTION OF DRAWINGS

Without restricting the full scope of this invention, the preferred form of this invention is illustrated in the following drawings in which:

FIG. 1 is a front view of the Leg Brace;

FIG. 2 is a right side view of the leg brace;

FIG. 3 is a back view of the leg brace;

FIG. 4 is a top view of the leg brace;

FIG. 5 is a bottom view of the leg brace;

FIG.6 is a left side view of the leg brace;

FIG. 7 is a front right perspective of the leg brace;

FIG. 8 is a view of the leg brace being used; and

FIG. 9 is a side view of the leg brace being used.

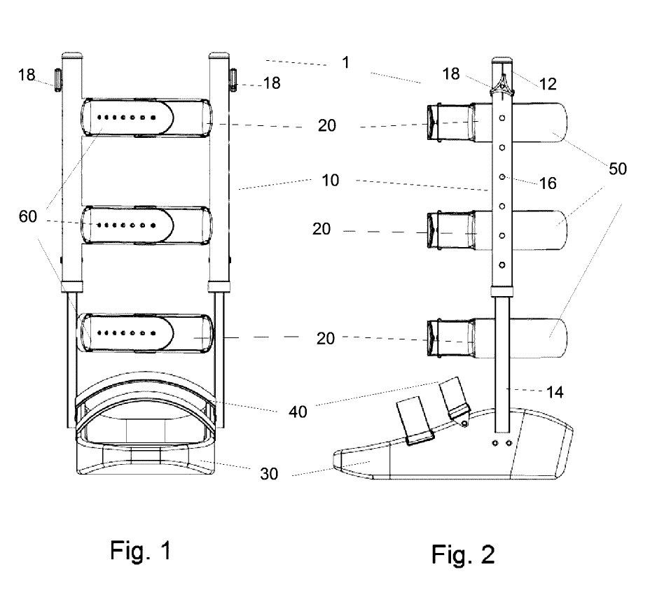

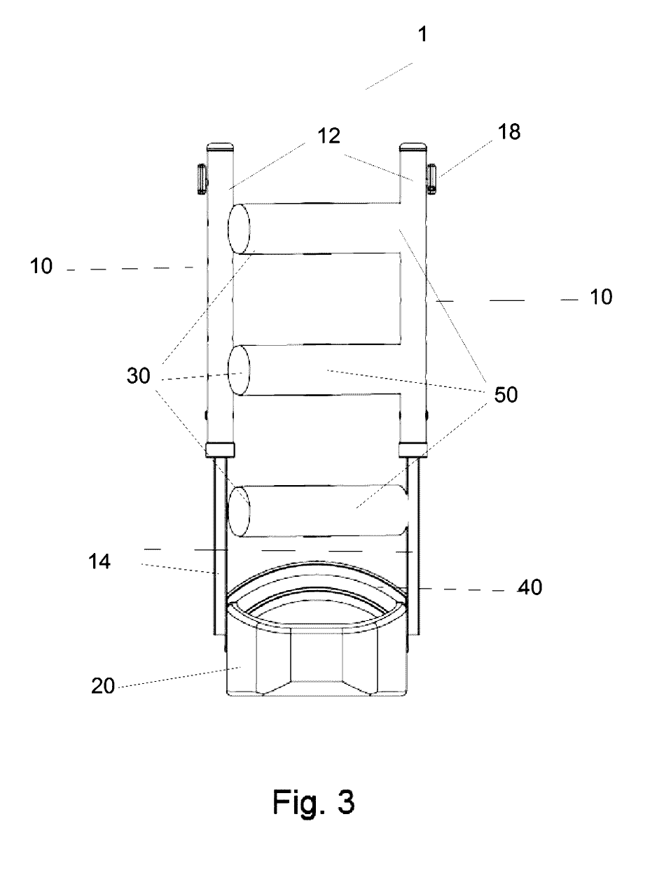

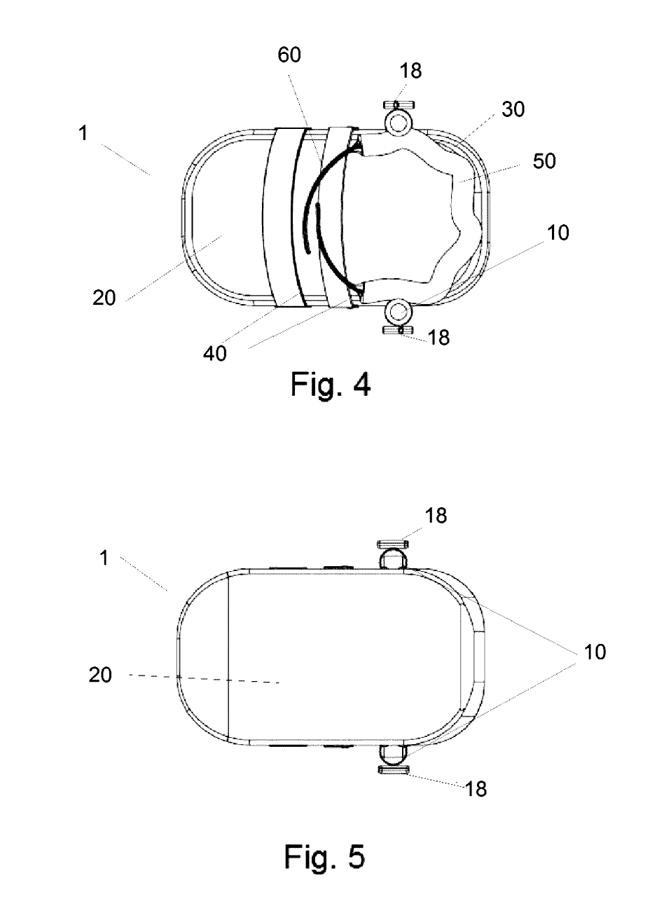

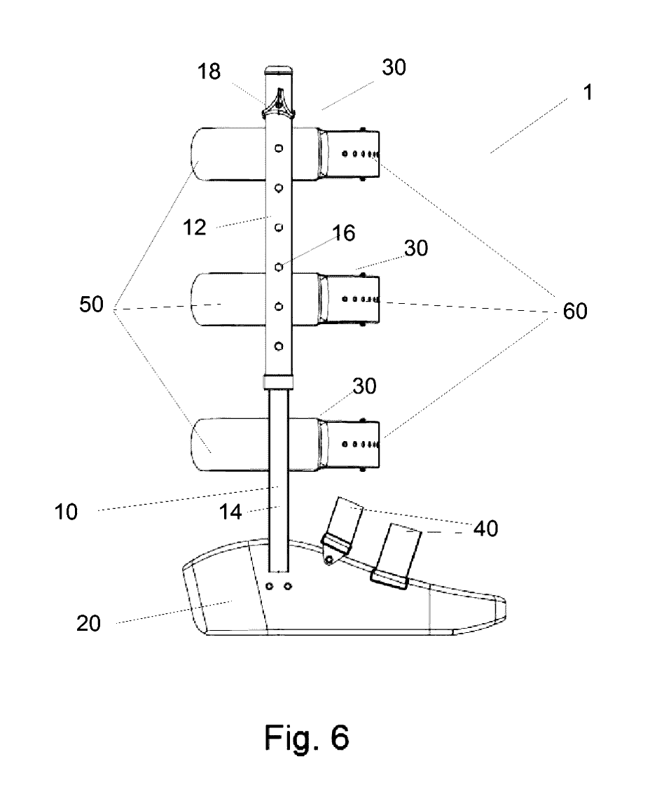

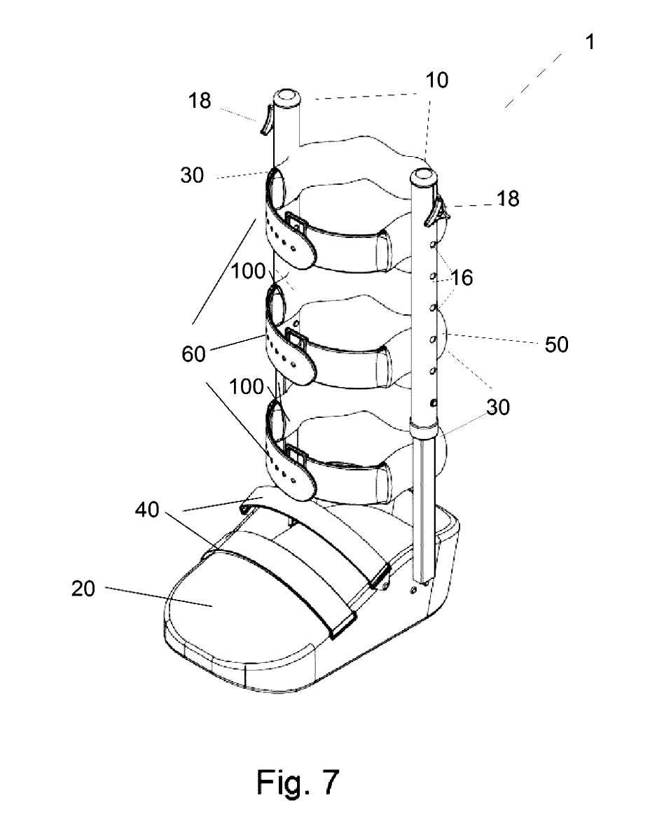

### DETAILED DESCRIPTION OF THE PREFERRED EMBODIMENT

There are a number of significant design features and improvements incorporated within the invention.

This proposed device is a leg brace 1 that can be used with either leg when that leg is injured.

The current invention is a leg brace 1 with key components being two adjustable lateral supports 10 connected to the footpad 20 with a plurality of support straps 30 that are connected between the lateral supports 10. These support straps 30 can be attached around the leg that needs to be support with an attachment means such as a buckle mechanism 60 or a hook and fastening system.

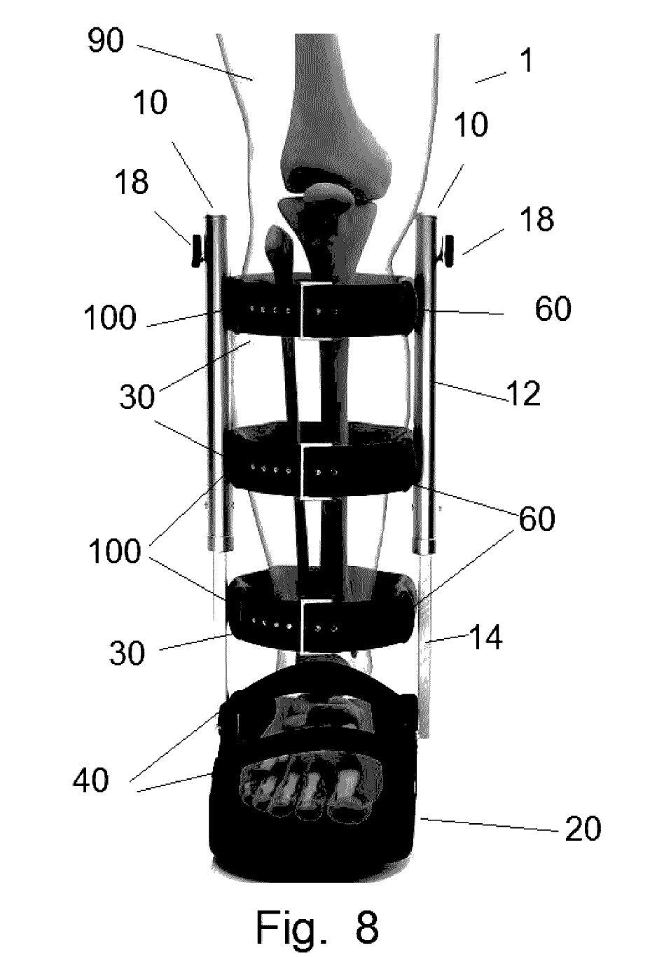

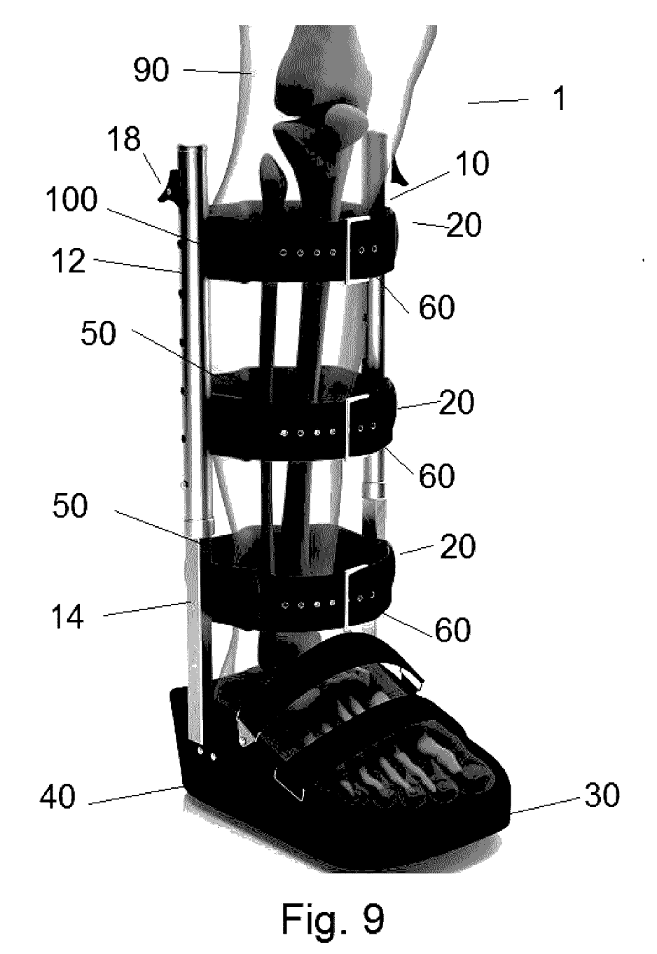

The footpad 20 would have a rocker bottom to support a person's natural gait and be made of a soft supportive material such as foam. A dense foam with a breathable liner is used in the preferred embodiment to provide comfort, dryness and support. The lateral supports 10 would connect to the footpad 20 one on each elongated side and located where the user's ankle would be positioned. The footpad 20 would have one or more footstraps 40 made of an elastic or plastic material. The footstraps 40 extend over the foot to hold the foot in the footpad 20 and shown in FIGS. 8 and 9. The top edge of the footpad 20 is contoured the fit a person's foot with a wide foot bed for stability.

The two adjustable lateral supports 10 are connected to the footpad 20. They consist of lateral support bar 14 and lateral support slide 12. The lateral support bar 14 fits inside the lateral support slide 12 with the slide 12 having the ability to move up and down on the bar 14 giving the lateral supports 10 the ability to be adjustable. Both the bar 14 and the slide 12 will have holes 16 in which a lateral support bolt 18 can be placed to hold the slide 12 and the bar 14 together at the desired height. The bar 14 and the slide 12 are made of a sturdy, strong, light weight material.

The leg brace 1 will have a plurality of support straps 20 that are connected between the lateral supports 10. These support straps 20 are attached around a user's leg 90, as shown in FIGS. 8 and 9, that needs to be support. The support straps 20 have an attachment means such as a buckle mechanism 60 or a hook and fastening system. The support straps 20 would have built in one or more air bladders 50 which would be inflated to the desired inflation with air using a built in air pump 100 allowing the proper support and comfort for the user. The user can adjust the amount of air in the bladder 50 to meet their needs and desires. The air pump 100 would be a finger pump where it is compressed using a person's fingers. The built in air pump 100 makes the leg brace 1 easier to use and fit properly. Having the air pump 100 attached makes it easier to use. There would be a release valve to release the air in the bladder 50. The support straps 20 are perpendicular to the lateral supports 10 and spaced apart based on the support needs of the user.

As to a further discussion of the manner of usage and operation of the present invention, the same should be apparent from the above description. Accordingly, no further discussion relating to the manner of usage and operation will be provided.

With respect to the above description, it is to be realized that the optimum dimensional relationships for the parts of the invention, to include variations in size, materials, shape, form, function and manner of operation, assembly and use, are deemed readily apparent and obvious to one skilled in the art, and all equivalent relationships to those illustrated in the drawings and described in the specification are intended to be encompassed by the present invention.

Therefore, the foregoing is considered as illustrative only of the principles of the invention. Further, since numerous modifications and changes will readily occur by those skilled in the art, it is not desired to limit the invention to the exact construction and operation shown and described, and accordingly, all suitable modifications and equivalents may be resorted to, falling within the scope of the invention.

I claim:

1. A leg brace comprising; a plurality of lateral supports connected to a footpad with a plurality of support straps attached to the lateral supports.

2. A leg brace according to claim 1 further comprising having the lateral supports being adjustable.

3. A leg brace according to claim 1 further comprising having the footpad have a rocker bottom.

4. A leg brace according to claim 1 further comprising having the footpad have one or more straps.

5. A leg brace according to claim 1 further comprising having the lateral support have a closing means.

6. A leg brace according to claim 5 further comprising having the closing means being a buckle.

7. A leg brace according to claim 1 further comprising having the support straps have a plurality of air bladders.

8. A leg brace according to claim 1 further comprising having a air pump connected to the air bladders.

9. A leg brace according to claim 8 further comprising having a air pump built in to the leg brace.

10. A leg brace according to claim 8 further comprising where the air pump is a finger pump.
